(19) United States
(12) Patent Application Publication
(10) Pub. No.: US 2023/0355117 A9
(43) Pub. Date: Nov. 9, 2023

(54) ENERGY EXPENSE DETERMINATION USING PROBABILISTIC INFERENCE

(71) Applicant: Yur Inc., San Mateo, CA (US)

(72) Inventors: Dilan Shah, San Francisco, CA (US); Cix Liv, San Francisco, CA (US); Max Meyers, Richmond, VA (US)

(73) Assignee: Yur Inc., San Mateo, CA (US)

(21) Appl. No.: 17/145,853

(22) Filed: Jan. 11, 2021

Related U.S. Application Data
(60) Provisional application No. 62/959,642, filed on Jan. 10, 2020.

(51) Int. Cl. A61B 5/024; A61B 5/00; A61B 5/11; G06F 3/01

(52) U.S. Cl. CPC A61B 5/024; A61B 5/7264; A61B 5/1128; G06F 3/011

(57) ABSTRACT

A mechanism for interpreting spatiotemporal data from augmented reality devices and virtual reality devices based on an analysis performed by one or more predictive machines. The predictive machines may implement machine learning techniques and/or algorithms to predict a heart rate for a user, from which user calorie expense can be calculated. In operation, a virtual reality or augmented reality device can receive positional data over time, for example derived from image data from a camera. The positional data can be averaged over time, and the averages can be rolled-up, and the averaged and rolled-up data can be fed into a predictive machine to generate a heart rate prediction. The heart rate prediction is used to generate a user heart rate, from which calories for the user's corresponding motion is determined.

### CROSS REFERENCE TO RELATED APPLICATIONS

The present application claims the priority benefit of U.S. provisional patent application No. 62/959,642, filed on Jan. 10, 2020, titled “Energy Expense Determination Using Probabilistic Inference,” the disclosure of which is incorporated herein.

### SUMMARY

The present technology, roughly described, provides a mechanism for interpreting spatiotemporal data from augmented reality devices, virtual reality devices, and other devices, based on an analysis performed by one or more predictive machines. The predictive machines may implement machine learning techniques and/or algorithms to predict a heart rate for a user, from which user calorie expense can be calculated.

In operation, a virtual reality or augmented reality device can receive positional data from one or more position tracking sensors over time as the user engages in an activity, for example with a corresponding environment. The positional data can be averaged over time, and the averages can be rolled-up, and the averaged and rolled-up data can be fed into a predictive machine to generate a heart rate prediction. The heart rate prediction is used to generate a user heart rate, from which calories for the user's corresponding motion is determined.

The present technology does not use a heart rate monitor to determine energy expense from the user. Rather, user energy expense is determined from user motions, captured from images or motion sensors in communication with a virtual, augmented, or other device, metadata, and from user biometric data. By not requiring a heart rate monitor, the user can enjoy activities, challenges, workouts, or other physical assertion without being inconvenienced with wearing a device that must be positioned to record heart rate data.

The present technology also automatically recognizes an exercise a user is performing, counts and reports the number of repetitions the user has performed, and can offer corrections for performing the exercise if it detects the user is not performing the exercise properly.

In embodiments, the present technology implements a method for automatically determining, using predictive learning, a heart rate for a user during a workout. The method includes receiving spatiotemporal data by a client application stored and executing on a client device. The spatiotemporal data indicates the spatial coordinates of a plurality of points associated with a user's body while the user is performing the workout, and the spatial data is received periodically to comprise spatiotemporal data. A heart rate is determined for the user during the workout using predictive learning and based on the spatiotemporal data. The heart rate is reported to the user during the workout by the client application.

In embodiments, a non-transitory computer readable storage medium has embodied thereon a program, the program being executable by a processor to perform a method for automatically determining, using predictive learning, a heart rate for a user during a workout. The method includes receiving spatiotemporal data by a client application stored and executing on a client device, the spatiotemporal data indicating the spatial coordinates of a plurality of points associated with a user's body while the user is performing the workout, the spatial data received periodically to comprise spatiotemporal data. A heart rate is determined for the user during the workout using predictive learning and based on the spatiotemporal data. The heart rate is reported to the user during the workout by the client application.

In embodiments, a system for automatically determining, using predictive learning, a heart rate for a user during a workout, includes a server having a memory and a processor. One or more modules are stored in the memory and executed by the processor to receive spatiotemporal data by a client application stored and executing on a client device, the spatiotemporal data indicating the spatial coordinates of a plurality of points associated with a user's body while the user is performing the workout, the spatial data received periodically to comprise spatiotemporal data, determine a heart rate for the user during the workout using predictive learning and based on the spatiotemporal data, and report the heart rate to the user during the workout by the client application.

### BRIEF DESCRIPTION OF FIGURES

FIG. 1A is a block diagram of an energy expense determination system within a virtual reality device.

FIG. 1B is a block diagram of an energy expense determination system within an augmented reality device.

FIG. 1C is a block diagram of an energy expense determination system that utilizes a device with a camera.

FIG. 2A is a block diagram of positional data capture and transmission devices.

FIG. 2B is a block diagram of a health module system.

FIG. 2C is a block diagram representing a predictive machine that includes a neural network.

FIG. 3 is an exemplary method for determining energy expense for a user using a predictive machine.

FIG. 4 is an exemplary method for determining average positional data.

FIG. 5 is an exemplary method for processing average positional data by a predictive machine to determine heart rate prediction.

FIG. 6 is another exemplary method for determining energy expense for a user using a predictive machine.

FIG. 7 is an exemplary method for classifying a user action.

FIG. 8 illustrates a user pose for a user's forearm.

FIG. 9 illustrates another user pose for a user's forearm.

FIG. 10 illustrates another user pose for a user's forearm.

FIG. 11 illustrates a user pose for a user's leg.

FIG. 12 illustrates a user pose for a user's shoulder.

FIG. 13 illustrates another user pose for a user's shoulder.

FIG. 14 illustrates a user pose for a user's leg.

FIG. 15 illustrates another user pose for a user's leg.

FIG. 16 illustrates another user pose for a user's leg.

FIG. 17 is a block diagram of a computing environment for implementing the present technology.

### DETAILED DESCRIPTION

The present technology, roughly described, provides a mechanism for interpreting spatiotemporal data from augmented reality devices, virtual reality devices, and other devices, based on an analysis performed by one or more predictive machines. The predictive machines may implement machine learning techniques and/or algorithms to predict a heart rate for a user, from which user calorie expense can be calculated.

In operation, a virtual reality or augmented reality device can receive positional data from one or more position tracking sensors over time as the user engages in an activity, for example with a corresponding environment. The positional data can be averaged over time, and the averages can be rolled-up, and the averaged and rolled-up data can be fed into a predictive machine to generate a heart rate prediction. The heart rate prediction is used to generate a user heart rate, from which calories for the user's corresponding motion is determined.

The present technology does not use a heart rate monitor to determine energy expense from the user. Rather, user energy expense is determined from user motions, captured from images or motion sensors in communication with a virtual, augmented, or other device, metadata, and from user biometric data. By not requiring a heart rate monitor, the user can enjoy activities, challenges, workouts, or other physical assertion without being inconvenienced with wearing a device that must be positioned to record heart rate data.

The present technology also automatically recognizes an exercise a user is performing, counts, and reports the number of repetitions the user has performed, and can offer corrections for performing the exercise if it detects the user is not performing the exercise properly.

The present technology can be used in a variety of applications. For example, the energy expense determination mechanism can be used to provide a virtual coach for activities and workouts, games involving physical movement, and other applications. In some instances, positional data captured for the user can be utilized, both in raw and processed form, to provide end-user specific physical recommendations in spatial environments through visual and/or audio cues.

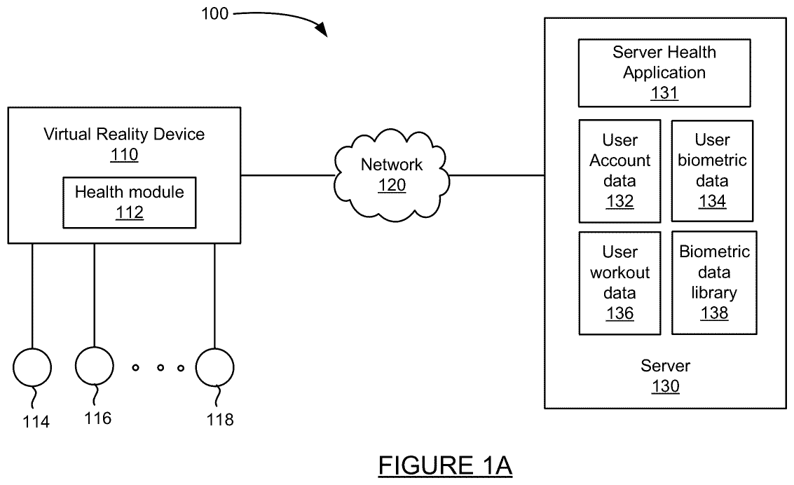

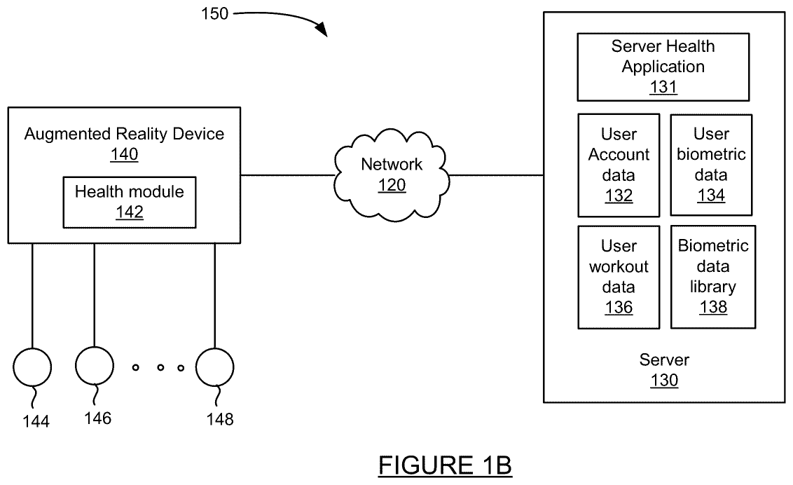

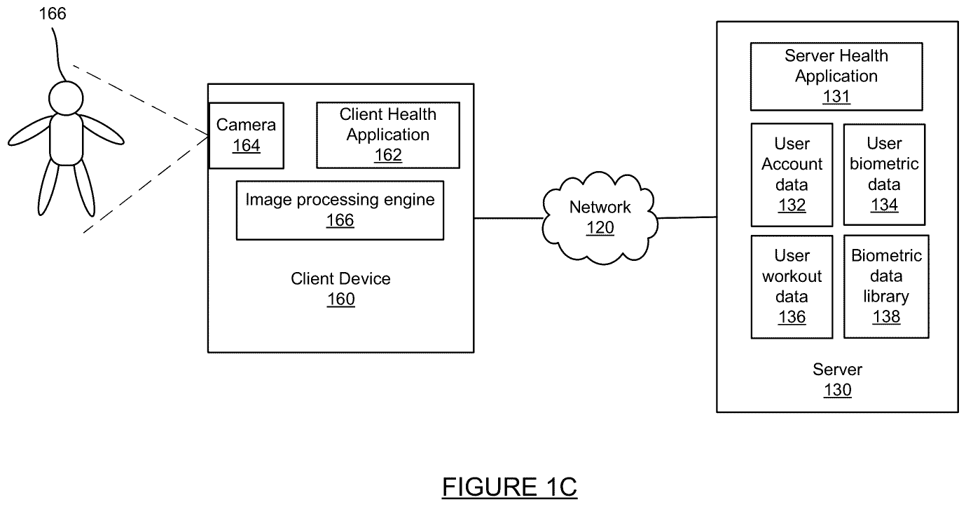

FIG. 1A is a block diagram of an energy expense determination system within a virtual reality device. System 100 of FIG. 1A includes virtual reality device 110, network 120, and server 130. Virtual reality device 110 may include one or more applications associated with the user performing a virtual activity in a virtual space. Virtual reality device 110 includes health module 112 and receives data from motion tracking sensors 114, 116 and 118. Although three motion tracking sensors are illustrated, any number of motion tracking sensors may be used with the present technology.

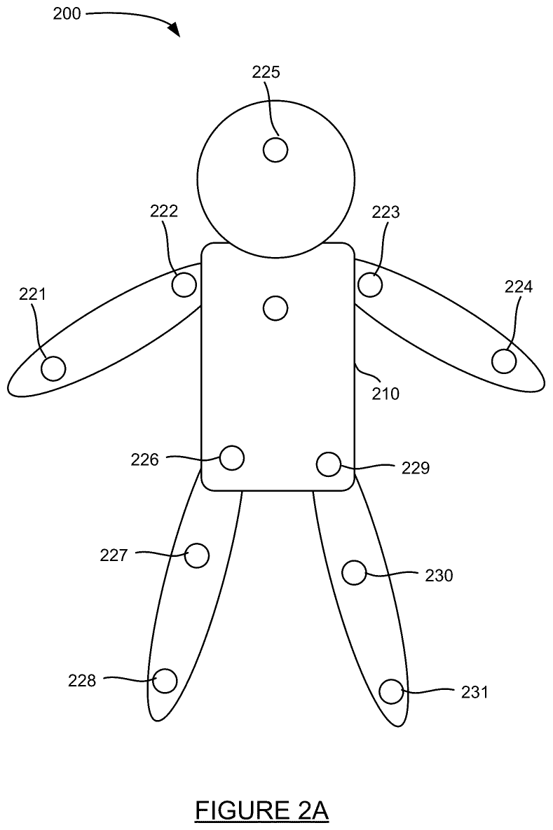

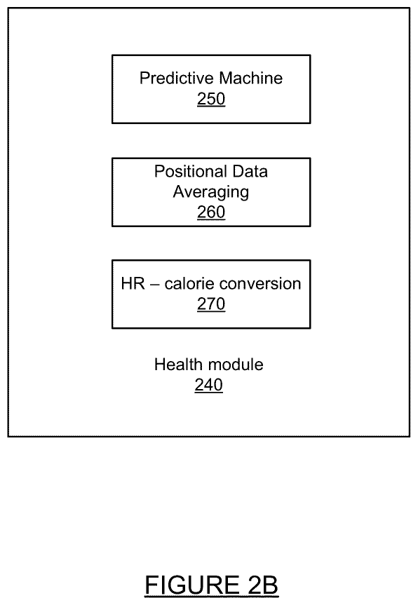

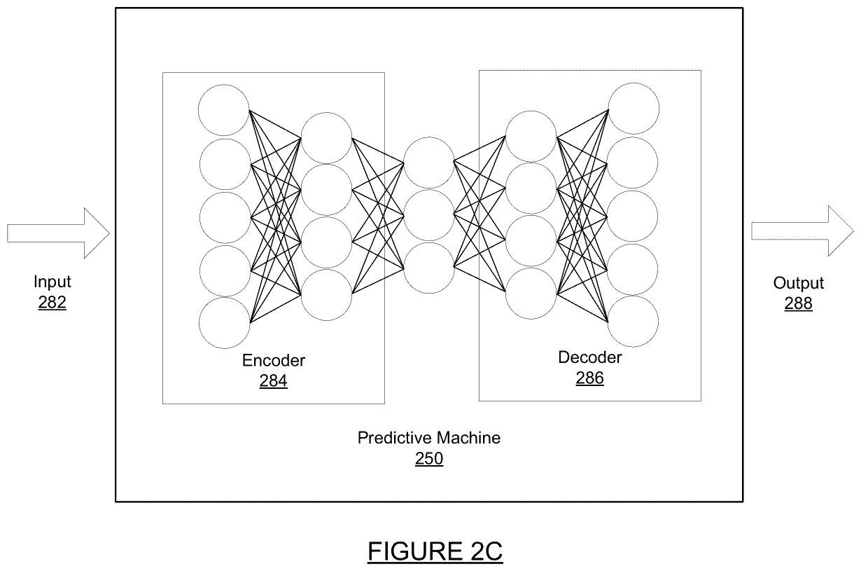

Health module 112 may be implemented as software and/or hardware on virtual reality device 110. In some instances, module 112 may be implement as code that interfaces with the native system of virtual reality device 110. Health module 112 may perform one or more operations that determine the energy expense of a user that utilizes virtual reality device 110 and may communicate with server 130 over network 120. The one or more operations may include averaging one or more sets of positional data received from one or more motion tracking devices, predicting a user heart rate indicator based on the averaged positional data, determining a heart rate for the user based at least in part on the heart rate indicator, and determining calories spent by the user based at least in part on the user's heart rate. The functionality of health module 112 is discussed in more detail herein, at least with respect to FIGS. 2A-6.

Network 120 may include one or more private networks, public networks, intranets, the Internet, wide-area networks, local area networks, cellular networks, radiofrequency networks, Wi-Fi networks, and any other network which may be used to transmit data. Device 110 and server 130 may communicate over network 120.

Server 130 can include server health application 131, user account data 132, user biometric data 134, user workout data 136, and biometric data library 138. Server health application 131 may receive and process requests from a client health application. In some instances, server health application 131 may perform one or more functionalities discussed with respect to a client health application, including but not limited to generating predictions, performing data averaging, and determining a heart rate. In some instances, server health application may handle requests and otherwise manage data such as the user account data, user biometric data, user workout data, and biometric data in library 138.

User account data 132 may include user data such as a usemame, password, user login information to one or more applications contained on device 110, and other account data. User biometric data may include biometric details for the user, such as a height, weight, date of birth, body mass index, lean body mass data, water percentage, and other data. User account data may also include a user maximum heart rate, user resting heart rate, user VO2 max data, and other health data. User workout data 136 may include details for one or more workout segments for the user. In some instances, a workout segment includes details for a particular workout characterized by a start time, end time, and other data.

Biometric data library 138 includes data obtained from the current user and/or other users relating to positional data and a corresponding energy expense associated with the data. In some instances, health module 112 may compare captured user positional data and biometric data to the biometric data library in order to determine an energy expense for the user's corresponding positional data.

FIG. 1B is a block diagram of an energy expense determination system within an augmented reality device. The system of FIG. 1B is similar to that of FIG. 1A except that the device is an augmented reality device rather than a virtual reality device. The health module 142, sensors 144-148, network 120, and server 130 of FIG. 1B operate similarly to those described with respect to FIG. 1A.

Though a virtual reality device and augmented reality device are discussed with respect to FIGS. 1A and 1B, it is understood that other devices can be used with the present technology as well. For example, health module 142 can be contained in any device that receives positional data from a number of attached or external sensors, such as a wearable fitness device or mobile phone or other device having a camera. The present technology is not intended to be limited to virtual reality and augmented reality devices.

The present technology can be used regardless of how the positional and/or movement data is provided to a health module. For example, the positional data can be captured and provided by motion tracking capture and transmission devices, analysis of one or more images or videos of a user that is moving, or some other movement data capture or generation system. The processing of the data and determination of a predicted heart rate and energy expense can be performed independently of how the data is captured.

The systems in FIGS. 1A-1B each include a health module in communication with a server. The distributed systems of FIGS. 1A-1B can use modelling techniques implemented by one more modules and/or software, including but not limited to predictive machines, machine learning, and other techniques, to predict a heart rate (i.e., an indication of a heart rate). In some instances, a server may maintain, update, modify, and otherwise manage the one or more predictive machines (e.g., modules or other code for performing at least a part of the technology described herein) and may provide the one or more predictive machines to VR device 110, AR device 140, or some other device, such that the receiving device can utilize the one or more predictive machines locally to perform at least part of the functionality described herein, including predicting the user's heart rate.

FIG. 1C is a block diagram of an energy expense determination system that utilizes a device with a camera. The block diagram of FIG. 1C includes client device 110, network 120, and server 130. Network 120 and Server 130 of FIG. 1C are similar components to the network and server discussed with respect to FIGS. 1A and 1B, and server 130 operate similarly as the servers from FIGS. 1A-1B.

Client device 160 includes client health application 162, camera 164, and an image processing engine 166. Client health application 162 of client device 160 may operate similarly as the client health applications of FIGS. 1A-1B.

Camera 164 may capture images of a user 166, for example while a user is performing an exercise or posing as part of an exercise. Images captured by camera 164 are processed by image processing engine 166. Image processing engine 166 may process the image data to identify joints such as knees, shoulders, and elbows, positions of body points such as the end of an arm or leg, movements, and may label parts of a user's body based on data within the images captured by camera 164. The data may then be provided to client health application 162 for processing.

FIG. 2A is a block diagram of positional data capture and transmission devices. In FIG. 2A, a user 210 may be fitted with one or more positional data capture and transmission devices. As shown in FIG. 2A, exemplary devices 221-231 may be placed on the user. In some instances, the devices may be placed on a user's arm, feet, body, head, and other locations. For purposes of discussion, the present technology may be discussed with respect to sensors 221 and 224 on the user's arms and sensor 225 on a user's head.

FIG. 2B is a block diagram of a health module system. The health module 240 of FIG. 2B provides more details for health modules 112 and/or 142 of FIGS. 1A-1C respectively. Health module 240 includes predictive machine 250, positional data averaging module 260, and HR-Calorie conversion module 270. Predictive machine 250 may include one or more modules that operate to predict a heart rate indicator based on positional data. The predictive machine can be implemented, in some instances, as a machine learning model having one or more machine learning algorithms that can be trained to predict a heart rate indicator from positional data. For example, the machine learning model can be implemented as one or more neural networks, algorithms that perform regression analysis, and/or other types of machine learning methodologies.

Positional data averaging module 260 can average and roll-up averaged data such as data retrieved from one or more images or data received from one or more motion sensing devices. For example, positional data averaging module 260 can average data received from one or more images, for example obtain an average for data received in 1 second windows. The positional data averaging module 260 can also “roll-up” averaged data by averaging sets of averaged data. For example, the positional data averaging module 260 can access the averages of the last n seconds (e.g., 2, 3, 6, 10, 120, and so forth) and take the average of the average values over the last n seconds.

HR-calorie conversion module 270 can calculate the calories burned per second based on a heart rate. In some instances, the average heart rate for a particular second is used to determine the calorie burn rate for that second for the user. In some instances, additional biometric data for a user, such as for example user age, weight, gender and so on, is also used in determining the calories burned for a user. More details for generating energy expense from a heart rate are discussed with respect to U.S. patent application Ser. No. 17/025,694, filed on Sep. 18, 2020, and entitled “Energy Expense Determination from Spatiotemporal Data.”

FIG. 2C is a block diagram representing a predictive machine that includes a neural network. The representative predictive machine block diagram of FIG. 2C includes an encoder 284 and a decoder 286. Input 282 is received by a predictive machine 250, encoded by the encoder 284, and then decoded by decoder 286. The decoder generates an output 288 which can then be further processed. In some instances, an encoder and decoder may have one or more levels of processing input data. The two levels are shown, one or more levels of encoding and decoding may be implemented within a predictive machine 250. Additionally, multiple sets of encoders and decoders, implementing multipole predictive machines, may be implemented within a predictive machine.

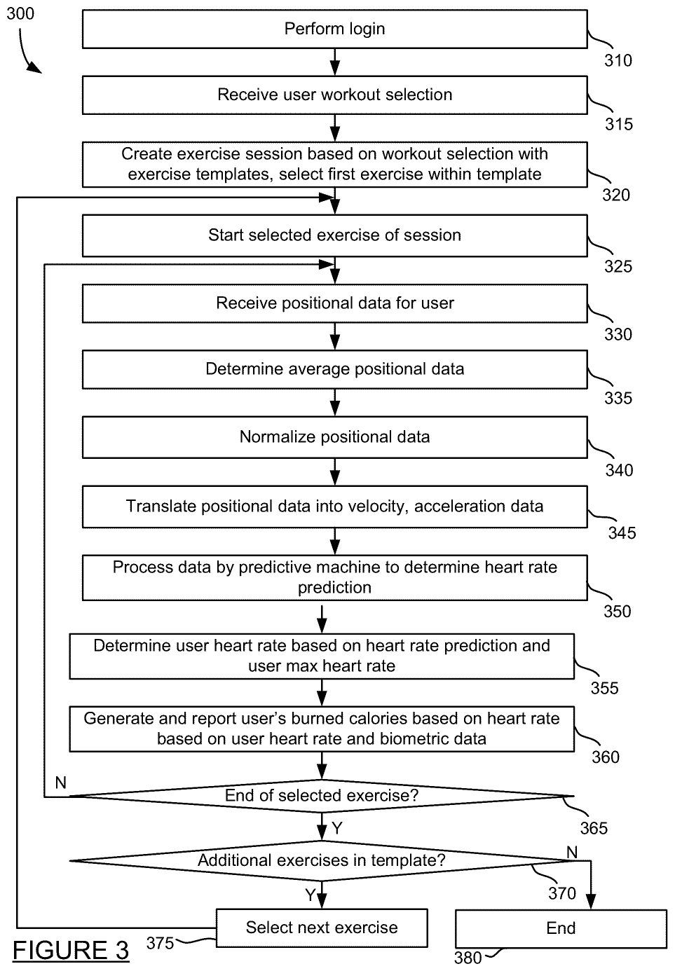

The present technology can determine a heart rate for a user using machine learning based predictive techniques to process positional data. The system can use machine learning techniques in several ways. In one instance, a machine learning system may process poses captured of a user and analyze positional data associated with the poses. The positional data may be analyzed with images associated with a state machine. An exercise may have several states that make up a state machine. As the user progresses through the exercise states, the state machine will progress through states associated with the exercise. Once an exercise is complete, the state machine returns to an initial state for that exercise, for example if there are more repetitions, or moves on to a new state machine associated with the next exercise. A method for predicting a heart rate using machine learning and state machines is discussed with respect to FIG. 3.

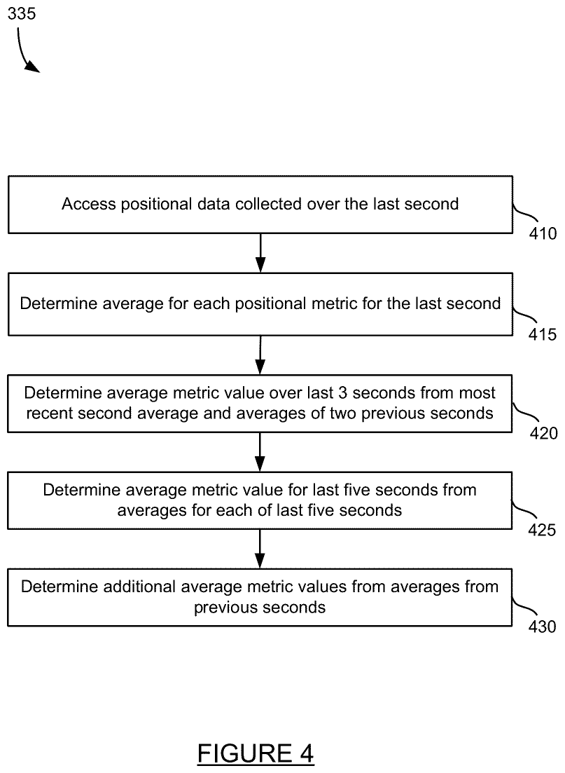

In another instance, the machine learning system may process poses captured by user, and analyze the positional data with respect to delegates and a prediction generated from the positional data or positional data derivative, for example angular velocity data. A method for predicting a heart rate using machine learning and predictions is discussed with respect to FIG. 4.

FIG. 3 is an exemplary method for determining energy expense for a user based on a predictive machine. First, login is performed at step 310. A user may login to one or more applications on a virtual reality or augmented reality device as well as software associated with the present technology. Login may include creating a new account for a user if needed, receiving login credentials for the user, confirming user login credentials are valid, and retrieving user biometric data. User biometric data may include, for example, a user's height, weight, a of birth, body mass index, and other biometric data associated with the user.

A user workout selection is received at step 315. In some instances, an interface may be provided to a user for selecting one of several workouts. A workout may include an activity, active game, a series of exercises, or some other physical activity. The input may be received through a touch display, microphone, joystick, or some other input.

An exercise session is created based on the workout selection at step 320. The exercise session may be associated with an exercise template. Each exercise template is associated with one or more exercises that are part of the workout. After creating an exercise session, a first exercise within the template is selected to begin the workout.

The selected exercise of the session starts at step 325. In some instances, beginning an exercise may include communicating by the client device that an exercise has started, and a user performing an activity, a workout task, or other exercise activity associated with the exercise. The client health application may provide some indication to a user that the exercise has started, such as sounds, video, and overlay of a display provided by camera, or some other indication. In some instances, the activity start indication may be the start of an application installed on a virtual reality or augmented reality device, such as an exercise application, physical therapy application, game application, or some other application that provides a physical workout, challenge, activity, or other physical experience. The indication may signify that a user play or experience space has been established for the user within a virtual or augmented reality space with a third-party application, and that the user is now able to engage in an activity.

Positional data for the user is received at step 330. In some instances, positional data can be retrieved by performing image processing of one or more images or a video of the user in motion. In this implementation, no sensors are required to be worn by the user. The image processing may include one of more identifying pixels associated with the user body, identifying pixels in the image/video associated with portions of the body for which to track movement, determining the movement of the user in the image between images and/or video frames, determining the units or length of movement based on camera data, image data, calibration information for the camera and environment, and providing the motion data for tracking.

In some instances, the positional data is received from one or more motion tracking sensors placed on the user's body. The data may be received wirelessly or via wired connections. In some instances, the positional data may be sampled periodically, such as for example between 60 to 100 Hz by the device 110 or 140

In some instances, there are three tracked points on a user—a tracking point on the head of a user, for example attached a tracking point on a headset worn by a user, and a tracking point at each hand of the user, for example a tracking mechanism attached to a glove, other user clothing, or directly attached to the user's hand. As a result of the three tracking points, the positional data can include position information for each of the three points, providing six degrees of freedom for each point. In this example, physical estimates of the tracking point locations, velocity, acceleration, orientation and/or overall body mechanics and activity can be determined from the positional data received for the three tracked points in six degrees of freedom.

Average positional data is determined at step 335. Determining average positional data can include determining an average value from one or more metrics received from one or more motion tracking devices for one or more set period of times. For example, a motion tracking sensor may provide data such as the linear velocity, linear acceleration, angular velocity, and angular acceleration. The motion tracking sensor may provide the linear and angular velocity and acceleration several times per second, such as for example between ten to seventy times per second. Averaging the data received from the particular motion tracking sensor may include determining the average linear velocity, average linear acceleration, average angular velocity, and average angular acceleration per second, per two seconds, per some fraction of a second, or an average for some other period.

Averages can also be rolled-up as additional averages. For example, if the average value is determined for the linear and angular velocity and acceleration every second for each of the last ten seconds, the average linear and angular velocity and acceleration can be determined for the last 10 seconds overall can also be determined. A rolled-up average can be determined for any number of past average calculations, such as for example, 3, 5, 30, 60, 120, and 300 seconds. More details for averaging positional data is discussed with respect to the method of FIG. 4.

The average positional data is normalized at step 340. The normalizing may be performed using any well-known averaging algorithm. In some instances, the normalization may be performed on positional data received at step 330 in addition to or in place of averaging the averaged positional data determined at step 335.

The averaged and normalized data may be translated into velocity and acceleration data at step 345. Translating the data into velocity and acceleration data may include taking data from multiple consecutive frames, calculating the velocity and acceleration data between identified points and add to identify joints, and recording of velocities, such as for example on angular velocity. In some instances, the data may be translated into linear velocity, linear acceleration, angular velocity, and angular acceleration. In some instances, the velocities and accelerations may be determined for different points on a user's body and joints (if generated from image data) or for each motion sensor (if using motion sensor output to determine positional data).

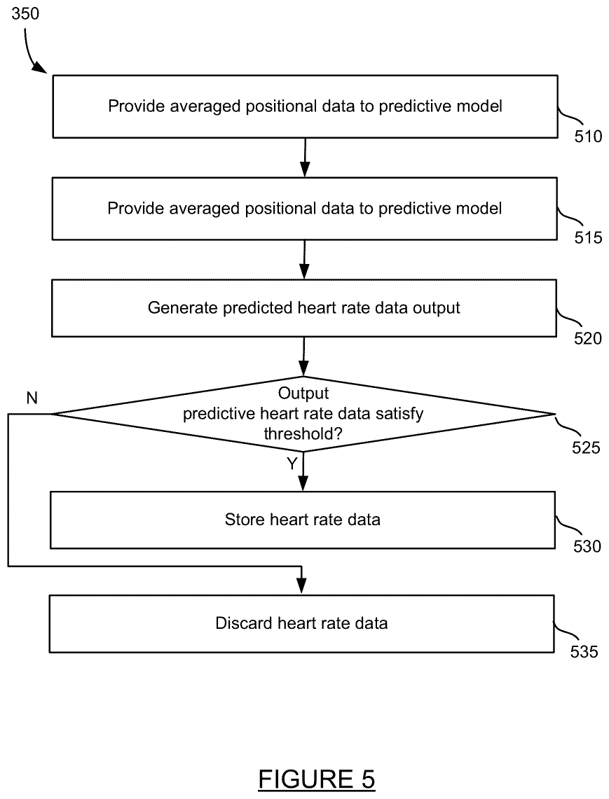

The translated data is processed by a predictive machine to determine a heart rate prediction at step 350. A predictive machine, trained by positional data input and heart rate data, is used to process positional data for the user. The predictive machine may use one more machine learning algorithms, including one or more algorithms that perform a regression analysis, to determine a heart rate prediction. More detail for predicting a heart rate for a user is discussed with respect to the method of FIG. 5.

A user heart rate is determined based on the predicted heart rate prediction at step 355. In some instances, the predicted heart rate provided by the predictive machine includes a multiplier, between 0 and 1, to apply against a maximum heart rate for the user. The maximum heart rate can be received from a user, determined based in user metric data (e.g., determining maximum user heart rate as 220−user age), or obtained in some other manner. Hence, if the predicted heart rate is 0.8, and the maximum user heart rate is 195, the user heart rate can be determined to be 0.9*195=176 (rounded up from 175.5).

In some instances, the predicted user heart rate can be used with other user heart rate data. For example, rather than applying the predicted heart rate to the user maximum heart rate, the predicted heart rate can be applied to a user resting heart rate.

A user's burned calories is determined based on the determined user heart rate, and the burned calories number is reported to the user at step 360. The calories can be determined based on any of several algorithms for determining calories burned for a user heart rate and other biometric data. For example, a formula for determining calories burned per second for a male is provided as:

Cₘₐₗₑ=((−55.0969+(0.6309×HR)+(0.1988×W)+(0.2017×A))/4.184)×60 calories/second

and for a female is provided as:

C_{female}((−20.4022+(0.4472×HR)−(0.1263×W)+(0.074×A))/4.184)×60 calories/second,

where HR is the user heart rate (beats/minute), W is the user weight (kg), and A is the user's age.

The calorie expense for the user may be reported through a dashboard, mobile device, or in some other manner. A dashboard may be provided through a network browser on a computing device, display of mobile device rendered by a mobile application on the mobile device (such as a cellular phone), or some other display mechanism. User energy expense may be updated in real time as user exercises and the updated energy expense is calculated. In some instances, historic workout data may also be reported, and may indicate the types of activities, duration, and calories burned for segments of workouts. Data from the dashboard can be stored to a health data store such as a database, a library of data accessed by a health application, such as “Google Fit” or “Apple Health” mobile applications, or some other electronic health record.

A determination is made at step 345 as to whether the user has completed the exercise. In some instances, a state machine determines the progress and/or status of a current exercise performed by a user. If a pose of a user is within an acceptable distance a state, the state increments to the next state. The process of comparing poses to an exercise state repeats until the exercise is completed at the last state. In particular, exercise may end when a user finishes the pose state machine. For example, a particular exercise can end when the particular activity, challenge, or game is complete. A user exercise may also end and if a user attempts to skip a state, such as for example by leaving the workout area. If the user has not ended the exercise, method of FIG. 3 returns to step 330 or additional positional data is received if the selected exercise has ended, and determination is then made as to whether there are additional exercises in the template at step 370. If additional exercises and the templates do exist, the next exercise is selected at step 375 and the workout continues at step 325. If the workout should end, then a user profile is updated, and workout history is recorded before the workout ends at step 380.

FIG. 4 is an exemplary method for determining average positional data. The method of FIG. 4 provides more detail for step 335 of the method of FIG. 3.

In the method of FIG. 4, steps are discussed that specify a specific period of time, such as for example accessing positional data collected “over the last second,” determining an average metric value “over last three seconds,” determining an average metric value “for the last five seconds,” and so forth. All references herein to a specific value of time, such as one second, the last second, three seconds, five seconds, and so forth are intended to be examples and mentioned for discussion purposes only. The present system is intended to be flexible in the periods of time during which data is collected and analysed. All references to specific lengths of time are intended as examples only and are not intended to be limiting.

Positional data collected over the last second is accessed at step 410. In some instances, positional data collected over some other time period, such as two seconds, 1.5 seconds, or some other period is accessed rather than data collected over the most recent one second.

An average for each motion tracking metric is determined for the most recent second at step 415. For example, for linear velocity positional data received from a left-hand motion tracking sensor by a virtual reality device, the linear velocity data values received over the last second are averaged into a single linear velocity data value for that second.

Average metric values for any number of seconds can be determined and used as input into the predictive machine. In some instances, the average of the last three seconds and last five seconds can be used, in addition to averages of other sets of data. The average metric value is determined over the last three seconds from the average value for the most recent second and the two previous seconds at step 420. The average metric value for the last five seconds is determined from averages for each of the last five seconds at step 425. Additional average metric values can be determined from average values of previous seconds at step 430.

FIG. 5 is an exemplary method for processing average positional data by a predictive machine to determine heart rate prediction. The method of FIG. 5 provides more detail for step 350 of the method of FIG. 3. Averaged positional data is provided as input to a predictive machine at step 510. The averaged positional data may include angular and linear velocity and acceleration data for several sensors that is averaged over a plurality of time periods.

The received averaged positional data is processed by the predictive machine at step 515. The predictive machine may include one or more algorithms that perform an analysis, such as for example a regression analysis, to determine a heart rate indicator based on the user's averaged positional data.

The predicted heart rate data output by the predictive machine is then generated and output at step 520. The predicted heart rate data output may include a high rate and a confidence score.

A determination is then made as to whether the output predictive heart rate data satisfies a threshold that step 525. A threshold may be applied to one or both of the generated did heart rate or confidence score. For example, a threshold of 20% may be applied to a generated heart rate, requiring that a subsequent heart rate is within a least 20% of a previous heart rate. Similarly, a threshold applied to a confidence score may require that the confidence score must be at least 0.7, 0.8 or 0.9, where is score of 1.0 is 100% confidence.

If the predictive heart rate data satisfies a threshold, the heart rate data may be stored at step 530. If the heart rate data does not satisfy one or more thresholds, the heart rate data may be discarded at step 535.

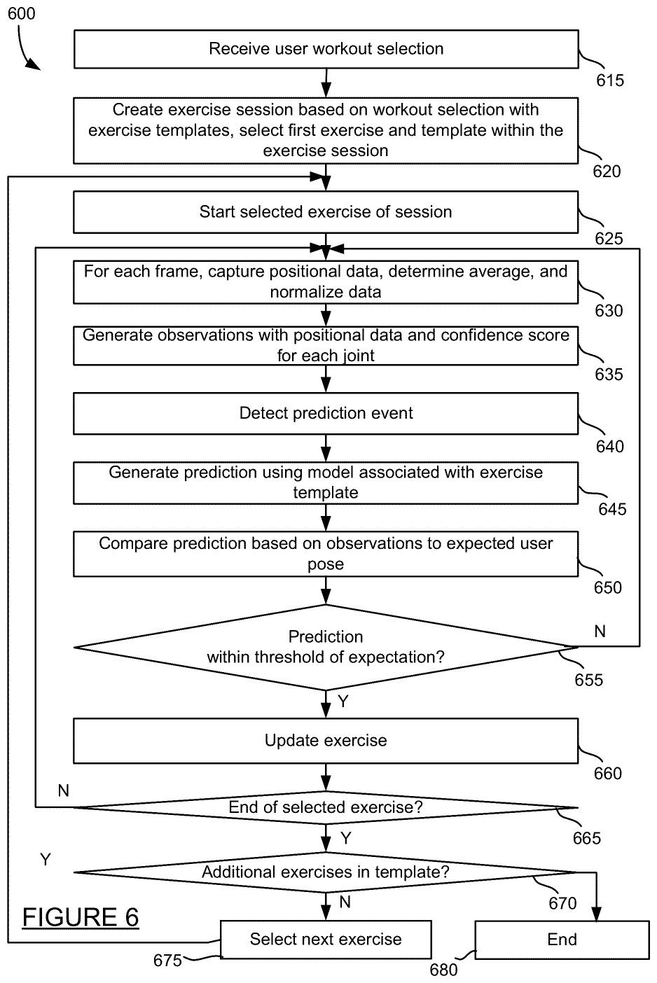

FIG. 6 is another exemplary method for determining energy expense for a user using a predictive machine. A user workout selection is received at step 615. An exercise session is then created based on the workout selection with exercise templates at step 620. The exercise session is associated with selected workout, associated templates, and parameters that define the session type, such as whether or not the session is a repeating session or fixed session. A first exercise and template are also selected from within the exercise session at step 620.

The selected exercise of the session is started at step 625. Once started, a camera on a client device captures images of a user performing the exercise. The images are processed, and the most recent captured image is labelled a delegate image. For each frame, positional data is captured, averages determined for the captured positional data, and the averaged data is normalized at step 630. In some instances, either the average data or the positional data, or both, may be normalized.

An observation is generated at step 635. The observation may be generated with positional data and a confidence score for each joint. Each generated observation, for example an observation for each frame or image captured by the camera over time, is stored for later processing when a prediction is to be generated.

A prediction event is detected at step 640. A prediction may be generated in response to a prediction event. A prediction event may be periodical, such as every 5 seconds. In some instances, the prediction event may be triggered after analysis of a current observation. For example, if the current observation appears to be in particular exercise position (such as a squat), a prediction event may be triggered.

A prediction is generated at step 645. The prediction may be generated using a model associated with the particular exercise template, and each exercise template may be associated with one or more different models. Hence, a prediction for a push-up exercise is generated from a different model than a prediction for a jumping jack exercise. Generating a prediction can include providing data associated with one or more observations, such as positions and confidence scores associated with the one or more observations, into the model associated with the particular exercise.

The output of the prediction is compared to the expected user pose at step 650. If the prediction indicates the user pose is within a threshold distance of the expected user position at step 655, the exercise is updated with the next expected pose at step 660 and the method continues to step 665. If the prediction is not within the threshold, the method of FIG. 6 returns to step 630.

At step 660, the system determined that the output of the model predicts that the user pose is as expected (for example, whether the user's body is further along an expected trajectory of an exercise). A determination is made at step 665 as to whether the current exercise is complete. If the current exercise is not complete, the method returns to step 630. If the exercise is complete, a determination is made as to whether there are additional exercises and the template at step 670. If there are additional exercises, a next exercise and corresponding template is selected at step 675 and the method of FIG. 6 returns to step 625. If there are no additional exercises and the template, the workout session ends at step 680.

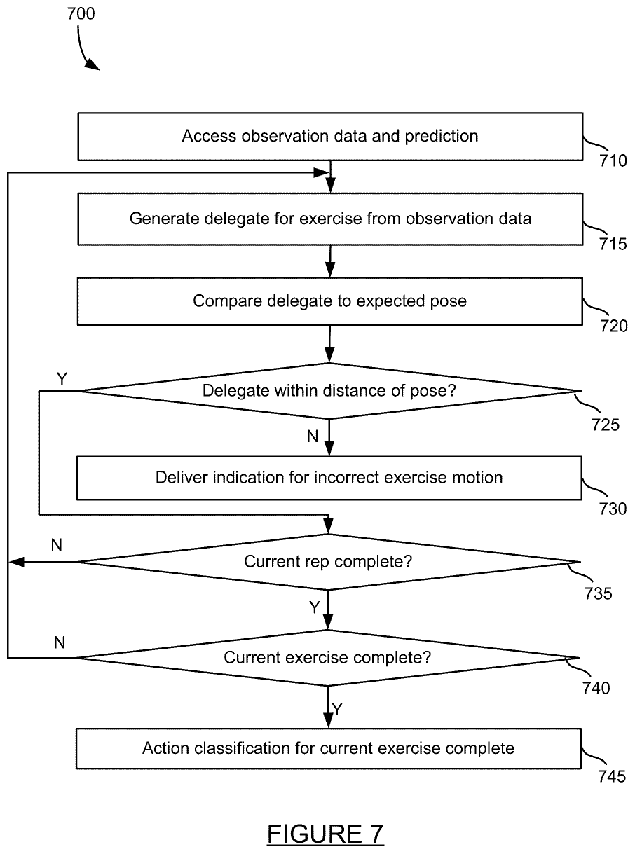

FIG. 7 is an exemplary method for classifying a user action. Observation data and prediction data may be accessed at step 710. A delegate for an exercise may be generated from the observation data at step 715. The delegate may be compared to an expected pose at step 720. If the delegate is within a distance of the expected pose at step 725, a user is considered on track to perform the exercise correctly and a determination is made as to whether the current rep is complete at step 735. If the delegate is not within a distance of the expected pose, an indication may be delivered to the user to correct the incorrect exercise motion or pose at step 730.

If the current rep is not complete at step 735, the method of FIG. 7 returns to step 715. If the current rep is complete, a determination is made as whether the current exercise is complete at step 740. If the current exercise is not complete, the method returns to step 715. If the current exercise is complete, the action classification for the current exercise is completed at step 745.

The repetitions (e.g., reps), exercise type, and exercise completion can be determined for a user for several exercise types. The exercise types include but are not limited to pulse lunges, side steps, kneel to stand, push-ups, push-ups on knees, good mornings, hip hinges, stance jacks, planks, shoulder squeeze, crab reach, hip lift march, airplane, high plank arm reach, squat side kick, plank body saw, v-up, v-sit, hip flow, glute kickback, alternating heel touch, helicopter, overhead squat, front squat, back squat, sumo deadlift, bent over row, deadlift, seated row, lat pulldown, pull up, chin up, negative pull up, negative chin up, wide grip pull up, standing incline chest press, squat to shoulder press, bicep curl, hammer curl, single arm tricep extension, seated shoulder press, bicep curl, hammer curl, single arm tricep extension, seated shoulder press, standing decline chest press, standing tricep extension, single arm incline chest press, underhand front raise, dumbbell overhead kneel to stand, dumbbell underhand front raise, elbows bent out bent over dumbbell row, high pull, face pull, single arm archer pull, kettlebell suitcase deadline, skierg, skierg butterfly, bench press, bent over row, and deadlift.

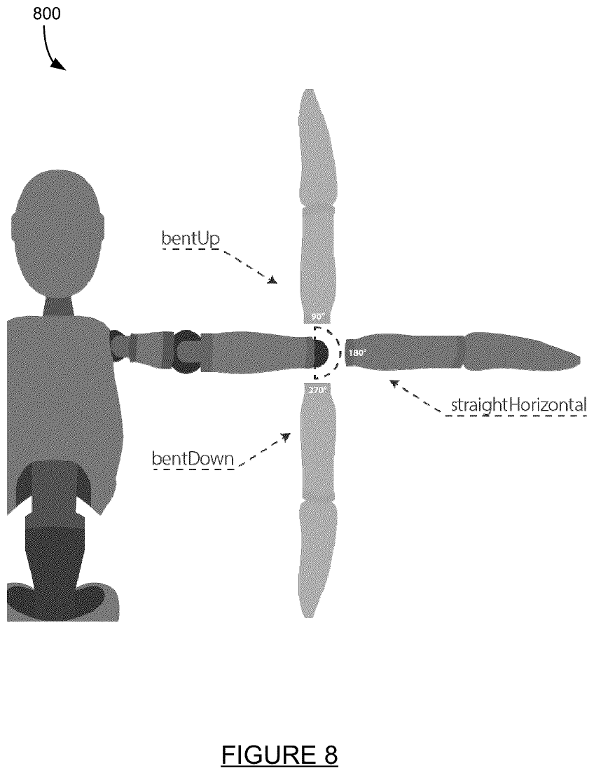

FIGS. 8-16 illustrate poses that a user may position in during one or more exercises. When comparing a user pose (i.e., pose data, delegate data, observation data, and/or other data) to an expected pose, the expected pose can be expressed as positional data relative to joints. The joints and limb positions are illustrated in FIGS. 8-16.

FIG. 8 illustrates a user pose for a user's forearm. The user pose of FIG. 8 illustrates user with an arm that can bend at the elbow such that the arm is bent up at 90° from the user's upper arm, extended straight and horizontal at 180° from the user's upper arm, or bent down at 270° from the user's upper arm.

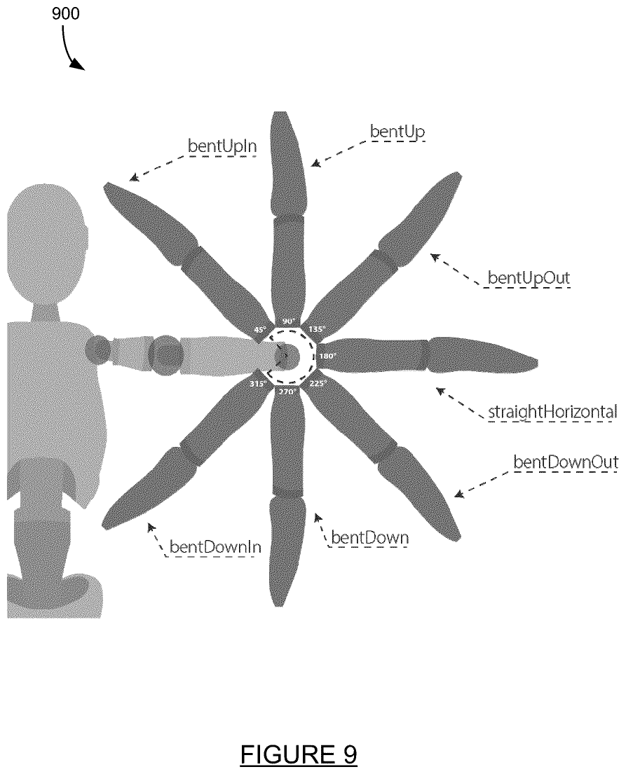

FIG. 9 illustrates another user pose for a user's forearm. The user pose of FIG. 9 illustrates additional positions for a user's arm with respect to the user's forearm, including positions in between each arm position of FIG. 8.

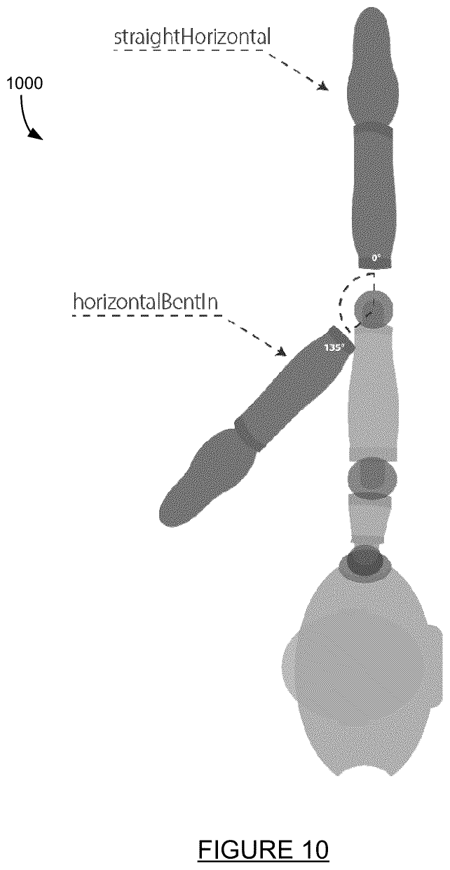

FIG. 10 illustrates another user pose for a user's forearm. FIG. 10 is a top view and illustrates a user's arm bending towards the user and a horizontal motion such that the arm is bent in towards the user 135° from being straight.

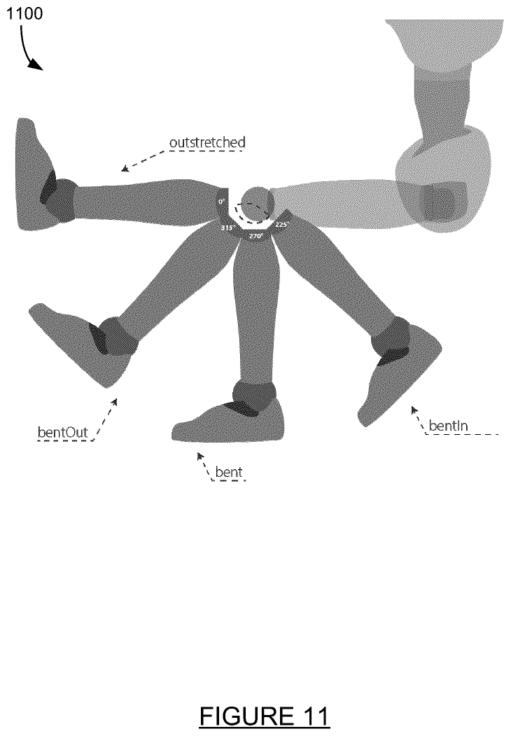

FIG. 11 illustrates a user pose for a user's leg. The pose of FIG. 11 illustrates a user's leg bent forward at the knee in different positions, ranging to being bent in towards the user.

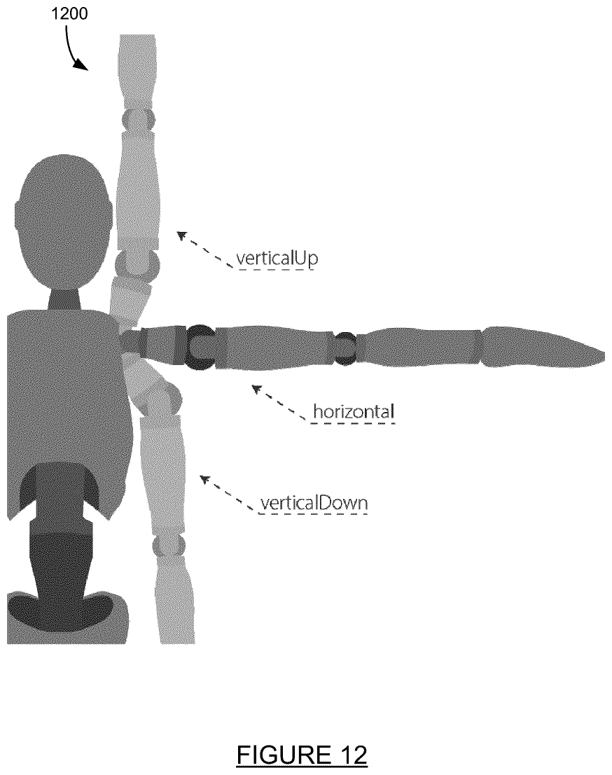

FIG. 12 illustrates a user pose for a user's shoulder. The pose of FIG. 12 illustrates an arm bending at a joint associated with a user shoulder, so that the arm can be extended up, down, or horizontal.

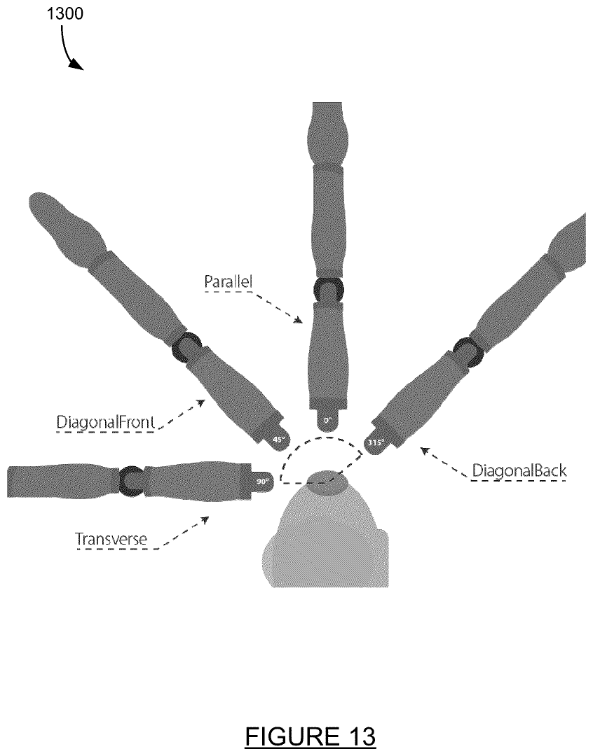

FIG. 13 illustrates another user pose for a user's shoulder. The pose of FIG. 13 illustrates a top view of the user, illustrating that a user's arm can be diagonally back, parallel, diagonally front, and transverse to the user.

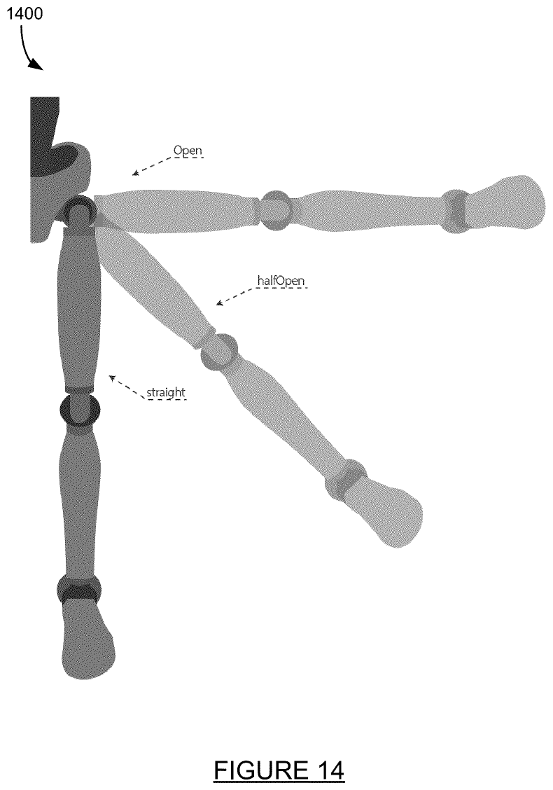

FIG. 14 illustrates a user pose for a user's leg. The pose of FIG. 14 illustrates a front view of a user's leg, indicating the leg can be vertically down, horizontally up, or in between.

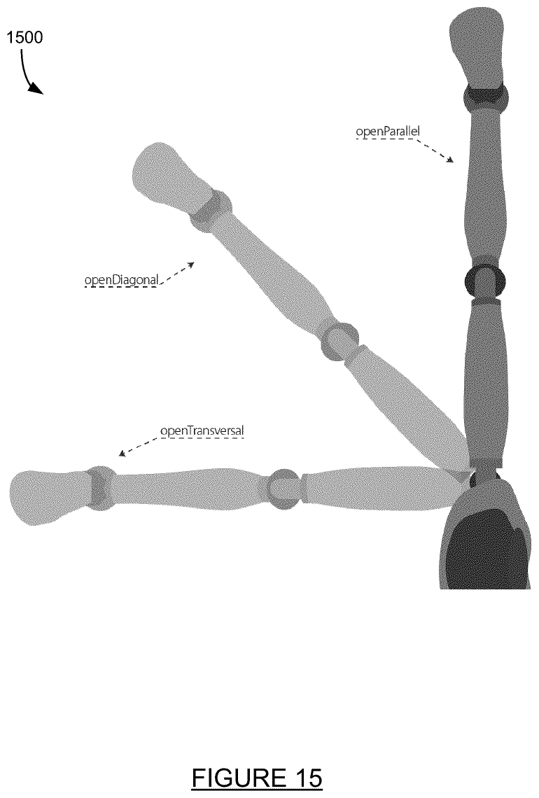

FIG. 15 illustrates another user pose for a user's leg. The pose of FIG. 15 illustrates a top view of a user, indicating that they can be stretched in front of the user, a word to the side of user, or in between.

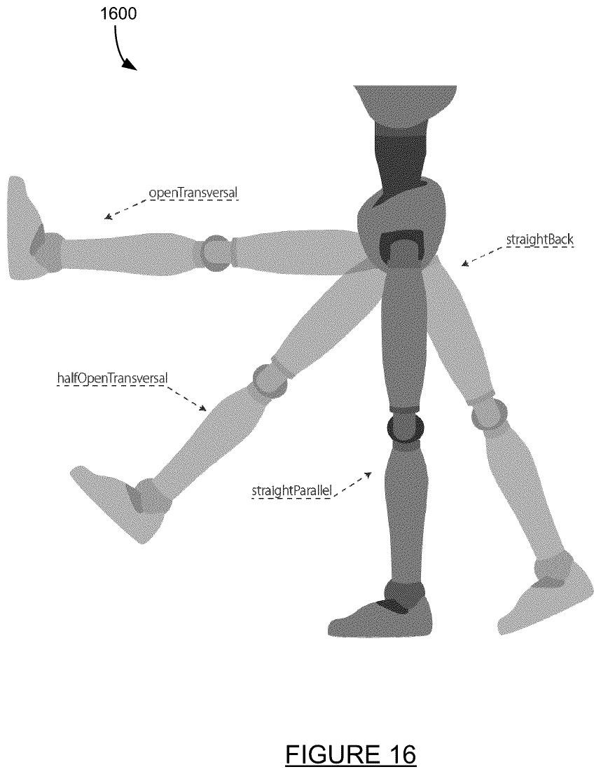

FIG. 16 illustrates another user pose for a user's leg. The pose of FIG. 16 is a side view of the user, showing that the leg can be brought forward to a horizontal position, kept as a straight position, brought back or brought forward.

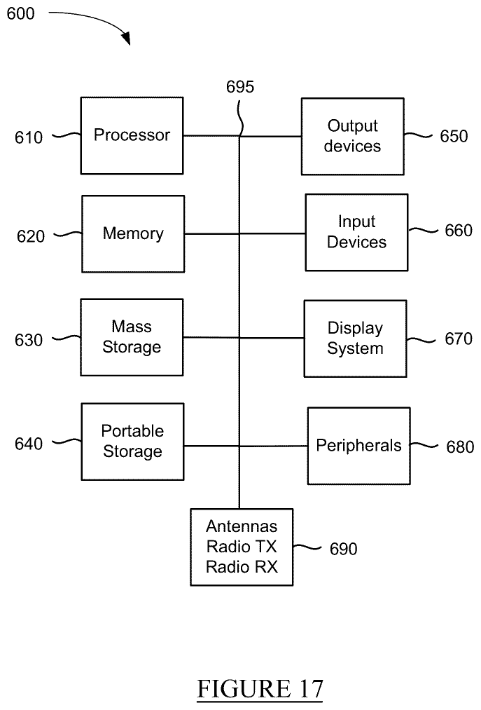

FIG. 17 is a block diagram of a computing environment for implementing the present technology. System 1700 of FIG. 17 may be implemented in the contexts of the likes of machines that implement a virtual reality device 110, server 130, augmented reality device 140, and client device 160. The computing system 1700 of FIG. 17 includes one or more processors 1710 and memory 1720. Main memory 1720 stores, in part, instructions and data for execution by processor 1710. Main memory 1720 can store the executable code when in operation. The system 1700 of FIG. 17 further includes a mass storage device 1730, portable storage medium drive(s) 1740, output devices 1750, user input devices 1760, a graphics display 1770, and peripheral devices 1780.

The components shown in FIG. 17 are depicted as being connected via a single bus 1790. However, the components may be connected through one or more data transport means. For example, processor unit 1710 and main memory 1720 may be connected via a local microprocessor bus, and the mass storage device 1730, peripheral device(s) 1780, portable storage device 1740, and display system 1770 may be connected via one or more input/output (I/O) buses.

Mass storage device 1730, which may be implemented with a magnetic disk drive, an optical disk drive, a flash drive, or other device, is a non-volatile storage device for storing data and instructions for use by processor unit 1710. Mass storage device 1730 can store the system software for implementing embodiments of the present invention for purposes of loading that software into main memory 1720.

Portable storage device 1740 operates in conjunction with a portable non-volatile storage medium, such as a floppy disk, compact disk or Digital video disc, USB drive, memory card or stick, or other portable or removable memory, to input and output data and code to and from the computer system 1700 of FIG. 17. The system software for implementing embodiments of the present invention may be stored on such a portable medium and input to the computer system 1700 via the portable storage device 1740.

Input devices 1760 provide a portion of a user interface. Input devices 1760 may include an alpha-numeric keypad, such as a keyboard, for inputting alpha-numeric and other information, a pointing device such as a mouse, a trackball, stylus, cursor direction keys, microphone, touchscreen, accelerometer, one or more cameras, and other input devices. Additionally, the system 1700 as shown in FIG. 17 includes output devices 1750. Examples of suitable output devices include speakers, printers, network interfaces, and monitors.

Display system 1770 may include a liquid crystal display (LCD) or other suitable display device. Display system 1770 receives textual and graphical information and processes the information for output to the display device. Display system 1770 may also receive input as a touchscreen.

Peripherals 1780 may include any type of computer support device to add additional functionality to the computer system. For example, peripheral device(s) 1780 may include a modem or a router, printer, and other device.

The system of 1700 may also include, in some implementations, antennas, radio transmitters and radio receivers 1790. The antennas and radios may be implemented in devices such as smart phones, tablets, and other devices that may communicate wirelessly. The one or more antennas may operate at one or more radio frequencies suitable to send and receive data over cellular networks, Wi-Fi networks, commercial device networks such as a Bluetooth device, and other radio frequency networks. The devices may include one or more radio transmitters and receivers for processing signals sent and received using the antennas.

The components contained in the computer system 1700 of FIG. 17 are those typically found in computer systems that may be suitable for use with embodiments of the present invention and are intended to represent a broad category of such computer components that are well known in the art. Thus, the computer system 1700 of FIG. 17 can be a personal computer, handheld computing device, smart phone, mobile computing device, workstation, server, minicomputer, mainframe computer, or any other computing device. The computer can also include different bus configurations, networked platforms, multi-processor platforms, etc. Various operating systems can be used including Unix, Linux, Windows, Macintosh OS, Android, as well as languages including Java, .NET, C, C++, Node.JS, and other suitable languages.

The foregoing detailed description of the technology herein has been presented for purposes of illustration and description. It is not intended to be exhaustive or to limit the technology to the precise form disclosed. Many modifications and variations are possible in light of the above teaching. The described embodiments were chosen to best explain the principles of the technology and its practical application to thereby enable others skilled in the art to best utilize the technology in various embodiments and with various modifications as are suited to the particular use contemplated. It is intended that the scope of the technology be defined by the claims appended hereto.

What is claimed is:

1. A method for automatically determining, using predictive learning, a heart rate for a user during a workout, the method comprising:
receiving spatiotemporal data by a client application stored and executing on a client device, the spatiotemporal data indicating the spatial coordinates of a plurality of points associated with a user's body while the user is performing the workout, the spatial data received periodically to comprise spatiotemporal data;
determining a heart rate for the user during the workout using predictive learning and based on the spatiotemporal data; and
reporting the heart rate to the user during the workout by the client application.

2. The method of claim 1, wherein the predictive learning includes one or more machines implemented on the client device that determine a heart rate and a confidence score.

3. The method of claim 1, wherein determining the heart rate includes generating a delegate for a user pose and comparing the delegate to a prediction.

4. The method of claim 3, further comprising determining if the delegate is within a threshold distance of the prediction.

5. The method of claim 3, further comprising generating the delegate from observation data, the observation data derived from image data of the user performing an exercise.

6. The method of claim 1, wherein the spatiotemporal data is received as data derived from image data of the user performing the workout.

7. The method of claim 1, wherein determining the user heart rate includes generating a modified heart rate based on the predictive learning generated heart rate and user biometric data that includes the user age, height, weight, and sex.

8. The method of claim 1, further comprising;
Identifying if a user is performing an exercise incorrectly; and
Providing an indication to the user to correct how the user is performing the exercise.

9. A non-transitory computer readable storage medium having embodied thereon a program, the program being executable by a processor to perform a method for automatically determining, using predictive learning, a heart rate for a user during a workout, the method comprising:
receiving spatiotemporal data by a client application stored and executing on a client device, the spatiotemporal data indicating the spatial coordinates of a plurality of points associated with a user's body while the user is performing the workout, the spatial data received periodically to comprise spatiotemporal data;
determining a heart rate for the user during the workout using predictive learning and based on the spatiotemporal data; and
reporting the heart rate to the user during the workout by the client application.

10. The non-transitory computer readable storage medium of claim 9, wherein the spatiotemporal data is received as data derived from image data of the user performing the workout.

11. The non-transitory computer readable storage medium of claim 9, wherein the predictive learning includes one or more machines implemented on the client device that determine a heart rate and a confidence score.

12. The non-transitory computer readable storage medium of claim 9, wherein determining the heart rate includes generating a delegate for a user pose and comparing the delegate to a prediction.

13. The non-transitory computer readable storage medium of claim 9, the method further comprising determining if the delegate is within a threshold distance of the prediction.

14. The non-transitory computer readable storage medium of claim 9, the method further comprising generating the delegate from observation data, the observation data derived from image data of the user performing an exercise.

15. The non-transitory computer readable storage medium of claim 9, wherein the spatiotemporal data is received as data derived from image data of the user performing the workout.

16. The non-transitory computer readable storage medium of claim 9, wherein determining the user heart rate includes generating a modified heart rate based on the predictive learning generated heart rate and user biometric data that includes the user age, height, weight, and sex.

17. The non-transitory computer readable storage medium of claim 9, the method further comprising;
Identifying if a user is performing an exercise incorrectly; and
Providing an indication to the user to correct how the user is performing the exercise.

18. A system for automatically determining, using predictive learning, a heart rate for a user during a workout, the system comprising:
a server including a memory and a processor; and
one or more modules stored in the memory and executed by the processor to receive spatiotemporal data by a client application stored and executing on a client device, the spatiotemporal data indicating the spatial coordinates of a plurality of points associated with a user's body while the user is performing the workout, the spatial data received periodically to comprise spatiotemporal data, determine a heart rate for the user during the workout using predictive learning and based on the spatiotemporal data, and report the heart rate to the user during the workout by the client application.

19. The system of claim 18, wherein the spatiotemporal data is received as data derived from image data of the user performing the workout.

20. The system of claim 18, wherein the predictive learning includes one or more machines implemented on the client device that determine a heart rate and a confidence score.
